(19) United States
(12) Patent Application Publication
(10) Pub. No.: US 2011/0076713 A1
(43) Pub. Date: Mar. 31, 2011

(54) Process of characterization of hepatocytes from goat

(76) Inventors: Vijaylakshmi Venkateshan, Hyderabad (IN); Naseem Begum, Hyderabad (IN); Aleem Ahmed Khan, Hyderabad (IN); Adarsh Kumar Capoor, Hyderabad (IN); Mohammed Aejaz Habeeb, Hyderabad (IN); Chittoor Mohammed Habibullah, Hyderabad (IN)

(21) Appl. No.: 10/539,889

(22) PCT Filed: Sep. 18, 2003
(86) PCT No.: PCT/IN2003/000316

§ 371 Date: Sep. 22, 2006

(30) Foreign Application Priority Data
Dec. 19, 2002 (IN) 1283/DEL/2002

(51) Int. Cl. C12Q 1/24

(52) U.S. Cl. 435/30

(57) ABSTRACT

A process for characterization of hepatocytes cells in goat comprising isolating goat hepatocytes and characterizing the isolated hepatocytes by biochemical and molecular techniques to demonstrate the hepatic functions.

### FIELD OF THE INVENTION

This invention relates to a process of characterization of hepatocytes from goat.

### BACKGROUND OF THE INVENTION

FHF is a clinical syndrome associated with a very high mortality rate, for which no satisfactory therapy is currently available, other than liver transplantation. FHF is a true medical emergency, which requires immediate therapeutic intervention. The treatment of FHF has been difficult due to the complex nature of physiological fractions performed by the liver. Liver transplantation is the only approach to improve survival in these patients. However, liver transplantation is a major surgical procedure, is very expensive, and requires prolonged inmumosuppression. It can be performed only at select advanced centers by highly skilled surgeons. Procurement of donor organs is also a major problem due to the shortage of human donors.

Hepatocyte transplantation appears to be an alternative to liver transplantation. Studies have been carried out to assess the efficacy of hepatocyte transplantation in acute liver failure in animal models such as rats, mice, and pigs, and have shown encouraging results (Sommer et al. 1979, Kawai et al. 1987, Gagandeep at al. 2000, Arkadopoulos et al. 1998, Allen and Soriano 2001, Malhi and Gupta 2001). Investigators have also demonstrated improvement in survival in human patients with FHF with allogeneic hepatocyte transplantation (Habibullah et al. 1994, Bilir et al. 2000). The data emerging from these experimental studies justifies the use of hepatocyte transplantation as a substitute to orthotopic liver transplantation.

The different sources of hepatocytes are human cadaver, human fetus and higher animals. Severe shortage of human cadaver livers, limits their use for therapeutic purpose. Human fetal hepatocytes from aborted or medically terminated pregnancies can be used, as fetal hepatocytes are less immunogenic, exhibit differentiated functions including albumin synthesis, bile formation, and urea cycle activity, and may provide metabolic support in FHF (Bayer et al. 1991, Rehman et al. 1993, Habibullah 1992, 1997). Habibullah et al. (1994) have reported that, intraperitoneal transplantation of human fetal hepatocytes resulted in the recovery of 3 out of 7 patients with acute liver failure. However the use of human fetal cells for transplantation is limited, as they are required in large numbers for a single transplantation.

The shortage of human donor organs has focused interest in the use of xenogeneic organs/cells for transplantation. Clinical interest in xenotransplantation (XT) and scientific research in this field have increased enormously (Reemtsma 1991). XT is considered promising because if it is successful, it can combat the shortage of human donor organs. The lives of many patients needing organ transplants may possibly be saved.

Pig xenografts have been considered as an alternative source of organs for transplantation (Niekrasz at al. 1992, Sach 1994). However, the use of pig organs/cells for human therapy has certain limitations. i) The presence of alpha-gal epitope (Gal alpha 1-3Gal beta 1-4GlcNAc—R) abundantly on pig cells, and its interaction with the human natural anti-Gal antibody, is considered to be the major obstacle in the way of pig to human xenotransplantation (Galili 1993). Incubation of pig cells expressing α-gal epitopes with human serum was found to induce complement mediated lysis of the cells as a result of the binding of anti Gal IgM molecules to α-gal epitope, followed by activation of complement (Good et al. 1992, Oriol et al. 1993, Sandrin et al. 1993). Studies in monkeys have farther indicated that, invivo binding of anti-Gal to α-gal epitopes on endothelial cells of pig xenografts results in complement mediated lysis of these cells, with ensuing collapse of the vascular bed and hyperacute rejection of the xenograft (Collins et al. 1994). ii) The risk of transmission of porcine endogenous retrovirus (PERV) to the recipients is also a major concern (Allan 1996, Patience at al. 1997, Speck at al. 2001).

### OBJECTS OF THE INVENTION

An object of this invention is to isolate the hepatocytes from goat.

Another object of this invention is to characterize the isolated cells to demonstrate the hepatic functions.

Further object of this invention is that the process is efficient and cost effective.

At the out set of the description which follows, it is to be understood that the ensuing description only illustrates a particular form of this invention. However, such a particular form is only intended as an exemplary embodiment and teaching of the invention and not intended to be taken restrictively.

### BRIEF DESCRIPTION OF THE INVENTION

According to this invention there is provided a process for characterization of hepatocytes cells in goat comprising isolating goat hepatocytes characterizing the isolated hepatocytes by biochemical and molecular techniques to demonstrate the hepatic functions.

Treating such isolated cells in a buffer medium and then subjecting it to a set up of characterization.

The shortage of human organs suitable for transplantation, coupled with recent advances in immunology has focused interest in the use of xenogeneic organs/cells for transplantation.

However, there are several barriers to xenotransplantation using pig organs/cells for human therapy, which include immune rejection, potential xenozoonosis and physiological incompatibilities. The major obstacle currently known, in the way of pig to human xenotransplantation is the interaction between the human natural anti-Gal antibody and the alpha-gal epitope (Gal alpha 1-3Gal beta 1-Glc Nac-R), which is abundantly expressed on pig cells.

The search for a potential source of organs/cells for the purpose of transplantation has diverted interest towards the use of goats. Goats have been extensively used in biomedical, medical, orthopedic, psychological, chemotherapeutic and physiological research. They have also shown promising results in xenobiotic oxidative metabolism. Goats have been used in xenotransplantation studies, in which the heart was transplanted from a pig let to a newborn goat. Newborn goats have been found to have low levels of xenoreactive natural antibodies. Blood samples collected after transplantation demonstrated a dramatic increase in anti-pig xeno-antibody titers and correlated with histological studies, demonstrating features consistent with delayed humeral rejection, including reactive vascular endothelial and perivascular stomal cells, marked capillary congestion, and interstitial hemorrhages. Scant to diffuse perivascular and interstitial infiltration of activated lymphoid cells occurred. Further, Macchiarini et al. (1999) have also demonstrated the presence of anti-pig xenoreactive antibodies in adult goats, resulting in hyperacute rejection of pig lung xenograft and death of the recipients within 7±3 hours after xenograft reimplantation. Further, they have shown that, pig left lung xenografts could provide prolonged and complete respiratory support after depletion of goat xenoreactive antibodies against pig cells, but they ultimately necrosed, once recipient xenoreactive antibodies returned to pretransplantation values. These reports demonstrate the presence of preformed natural antibodies in the goat, which simulates the human system.

Goats are safe to handle, easy to domesticate, have big litters, are easy to feed and their usage in the xenobiotic oxidative metabolism is also promising. Further, there areno studies invivo or invitro to demonstrate the feasibility of goat hepatocytes in the treatment of acute liver failure. In this study we have chosen goat as a higher animal model to investigate its potential application as a xenogenic source of hepatocytes in the management of liver diseases such as acute liver failure.

The primary requirement of the cells used in transplantation is the preservation of their viability and metabolic fractions so that they prevent/decrease hepatic encephalopathy when transplanted in patients with acute liver failure. So, in the first phase of our study, we have made an attempt to assess the viability and metabolic functions of goat hepatocytes and have compared with those of human fetal (28-36 weeks) and pig hepatocytes. We have used 28-36 weeks human fetuses, as they reflect adult liver like characteristics in their structure and functions.

In the present study, the two-step collagenase digestion method was adopted for the isolation of hepatocytes in all the three groups. The cell yield obtained from the pig and human fetal livers in our conditions is comparable with the yield as reported by other investigators (Nordlinger et al. 1985, Habibullah et al. 1990). This method of isolation of hepatocytes gave a good cell yield and thus appears to be a suitable method in providing isolated hepatocytes as a biological tool for the study.

Experimental data generated during the present study showed a viability of 90-95% by trypan blue dye exclusion technique in the hepatocytes isolated from goal, pig and human fetus. The data is similar with the findings of other investigators. Seglen (1976) reported a viability of 80-95% by the two-step collagenase digestion method. Klaunig et al. (1981) using the two-step collagenase perfusion method reported 93-95% viability.

The viability of the hepatocytes by MTT assay and the values have been found to be comparable among all the three groups. The assay, which requires no labeling of target cells, provides indirect estimates of the mitochondrial oxidative processes of living cells. MTT assay has been considered to be an accurate, simple and time saving method and good correlation in the results has been shown with other methods for testing the viability of the cells, such as thymidine incorporation, trypan blue dye exclusion, and fluorescein diacetate (FDA) methods.

LDH leakage, Na⁺K⁺ATPase activity and lipid peroxidation are the markers of cellular viability and membrane integrity. Experimental, evidences support cytosolic LDH as a marker of viability. In the present invention, the percentage LDH leakage was also comparable among the three groups.

Na⁺K⁺ATPase activity also did not reveal any significant difference among goat, pig and human fetal hepatocytes. Available reports confirm that, cellular death is directly related to the decrease in Na⁺K⁺ATPase activity. Na⁺K⁺ATPase activity has been used as a marker of cellular viability and membrane integrity in isolated hepatocytes.

Lipid peroxidation, which has been determined through the formation of Malondialdehyde (MDA), is a useful means to assess tissue damage. In present invention the MDA levels, which reflect the membrane integrity of the cells, did not show any significant difference among the three groups studied.

Ureagenesis is an important liver detoxifying function. Investigators have studied the urea production capacity and urea cycle enzymes in the fetal liver tissues and isolated and cultured hepatocytes. Ureagenesis has been measured as a differentiated hepatocyte fraction while using the hepatocytes in a hybrid bioartificial device, as it is necessary that they maintain the detoxification function for the management of FHF. Cytochrome P450 enzymes play a major role in the metabolism of a wide array of endogenous and exogenous (Xenobiotic) toxins. Detoxification of benzodiazepine-like substances by hepatocytes is of particular importance due to the putative role of these substances in the development of hepatic coma. In the present invention, ureagenesis, as measured by ammonia loading test did not show any significant difference among the three groups.

Thus, the present experimental details on viability, membrane integrity and detoxifying capacity of goat, pig and human fetal hepatocytes show that they are comparable in their metabolic fractions.

The cytotoxic effect of human serum on goat hepatocytes has been evaluated as compared to its effect on human fetal and pig hepatocytes. Several studies have demonstrated that human serum has a cytotoxic effect on pig cells, including endothelial cells, kidney cells, and hepatocytes. It is considered that this effect is due to the binding of preformed natural antibodies to the pig cells and subsequent activation of complement. Thus, transplantation of organs/cells from pigs to humans would lead to hyperacute rejection.

In the present invention, cytotoxicity of human serum on goat and pig hepatocytes was studied by MTT assay. The results showed about 36% lysis of pig hepatocytes on incubation with human serum. We used human fetal hepatocytes as control, which showed about 90% viability with human serum. Goat hepatocyte also recorded a viability about 90% and was comparable to that of human fetal hepatocytes on incubation with human serum.

Thus, the present study has demonstrated that goat hepatocytes were comparable to that of human fetal hepatocytes, in not mediating lysis of the cells, after incubation with normal human serum, unlike pig hepatocytes, which showed significant lysis of the cells. These results have encouraged us to further investigate the effect of antibody-depleted human serum on goat hepatocytes, in comparison to pig hepatocytes, which would indicate whether antibody mediated lysis is taking place in the goat hepatocytes.

The results using antibody depleted human serum have shown comparable viability of goat hepatocytes on incubation with normal as well as antibody depleted human serum, whereas the percentage of viability of pig cells with antibody depleted human serum was about 82% as compared to about 64% viability with normal human serum which was statistically significant (p<0.01). Our results of antibody depleted and normal human sera on pig cells are in line with the results of other investigators who have also shown a significant increase in viability of pig islets on incubation with antibody depleted human serum compared to normal human serum. The data showing significant loss of viability of pig cells with normal human serum compared to antibody depleted serum, could probably be due to the binding of anti-Gal antibody in human serum with α-gal epitopes present on pig cells, and subsequent activation of complement, resulting in lysis of cells. In our study, the viability of goat cells on incubation with normal human serum and antibody-depleted serum have shown comparable results, which may probably be due to the absence of anti-Gal antibody—α-gal interaction in the goat cells and human serum. However, this is a preliminary work, which has to be further confirmed by other methods, such as lectin binding assays and molecular techniques using monoclonal antibodies. Based on our results, which has shown comparable metabolic functions of goat hepatocytes with those of pig and human fetus, as well as the compatibility of goat hepatocytes with human serum, teat hepatocytes have been used in further experiments. The invivo experiment was carried out to prove the efficacy of transplanted goat hepatocytes in D-galactosamine-induced acute failure model such as wistar rat. The group I animals served as controls. Group II animals received only D-galactosamine and the group III animals received goat hepatocytes 24 hours after D-galactosamine liver injury. In the present study, groups III animals demonstrated 90% survival as compared to 0% in-group II. Further, the biochemical impairments that developed following acute liver injury reverted back to normal in animals, which received the hepatocytes.

Prothrombin time (P.T) is a sensitive indicator of liver damage. We observed that the P.T. value was exceeding one minute by animals 24 hours after D. galactosamine injection and the animals were going into coma between 24 to 36 hours. We also observed that hepatocyte transplantation could not increase the survival of the animals when P.T. value exceeded 2 minutes. Our data showed that, there is a significant increase in P.T and blood ammonia levels between 24 to 36 hours (p<0.001) after D-galactosamine liver injury. Following hepatocyte transplantation, P.T. and blood ammonia decreased and reached the normal value by Day 10 in-group III animals. Similarly, other liver function tests, such as total serum bilirubin and ALT activity, which were significantly high after 24 hours of D-galactosamine injection, reached the normal values on day 10 and 20 respectively. However, serum albumin levels did not show any significant difference following D-galactosamine liver injury and hepatocyte transplantation.

These liver specific markers were studied so as to assess the status of the liver before and after hepatocyte transplantation, in order to gain hither insight into the fact, whether these transplanted hepatocytes were extending any support towards the reversal of the liver failure.

Histopathological studies following liver injury and after hepatocyte transplantation, up to one month of follow up, showed initial hepatic necrosis (massive necrosis between 24-36 hours) with gradual recovery after hepatocyte transplantation and the attainment of normal histology by the end of one month (Group III animals).

This study infers that, transplantation of goat hepatocytes intraperitoneally in D-galactosamine induced liver allure in Wistar rats normalizes their liver functions and leads to recovery of their normal histology. Thus, the feasibility of the transplanted goat hepatocytes in bringing about reversal of the hepatic damage in FHF rat model has been demonstrated in the present study. This is a preliminary work, which is of clinical interest and requires further exhaustive studies in higher animal models before considering for possible clinical transplantation in humans, in future.

### EXAMPLES

Materials and Methods

Goat:

The use of animal tissues for experimentation was approved by the Committee for the Purpose of Control and Supervision of Experiments on Animals, Ministry of Social Justice and Empowerment, Government of India vide registration No.178/CPCSEA. The goats (Capra hircus, Deccani breed) used for the study were apparently healthy animals, six months to one year old (15-20 Kgs), and were obtained from a veterinary center. They were housed according to Laboratory animal care requirements. They were fed on grass, tree fodder and unlimited water. They were used for the experiments after ruling out the presence of common bacterial diseases, mycoplasma and brucellosis.

Pigs:

The out bred adult pigs used for the study were also collected from a veterinary center, and examined by a veterinarian to monitor their status of health. They were housed according to Laboratory Animal Care Requirements. They were provided with commercial feed and unlimited supply of water. The livers were collected in cold and sterile conditions after slaughtering the animals. The procedure for the isolation of pig hepatocytes was similar to that of goat hepatocytes as described in section 3.4.2.

Human Fetuses:

The use of human fetal tissue for experimentation was approved by the ethical committee of the Indian council of Medical Research, New Delhi, Government of India. Human fetuses (28-36 wks) were obtained from the local maternity hospital as a result of spontaneous abortions with the consent of parents or guardians.

Isolation of Hepatocytes:

Hank's buffer: It was prepared by dissolving 8 gms of NaCl, 400 mg of KCl, 60 mg of Na2HPO₄2H₂O, and 60 mg of KH₂PO₄2H₂O in 950 ml of double distilled water. The pH was adjusted to 7.2 with 1 M NaHCO3. The volume was then made up to 1000 ml with distilled water, autoclaved at 15 psi for 25 minutes, and later, penicillin (100 n/ml) and streptomycin (100 μgm/ml) were added.

Hank's Medium: It was prepared by mixing 8 gms of NaCl, 400 mg of KCl, 200 mg of MgSO₄7H₂O, 60 mg of NaH₂PO₄2H₂O, 60 mg of KH₂PO₄2H₃O, 1 gm of glucose, and 140 mg of CaCl₂2H₂O {Glaxo India Ltd.} and dissolving in 950 ml of double distilled water. The pH was adjusted to 7.2 with 1 M NaHCO₃. The volume was then made up to 1000 ml with double distilled water, autoclaved at 15 psi for 25 minutes and later, penicillin (100 u/ml) and streptomycin (100 μgm/ml) were added.

Collagenase solution (0.025%): 25 mg of Type IV collagenase {Sigma Chemical Co. USA} was dissolved in 100 ml of Hank's medium.

Goat and Pig:

The animals were sacrificed and livers were collected in cold and sterile conditions. They were processed for the isolation of hepatocytes in a laminar flow unit under aseptic conditions by the method of Habibullah et at 1990, with slight modifications. The liver was perfused extensively with Hank's buffer through the portal vein, using a polythene catheter, until it became colourless. Collagenase solution (15-20 ml) was passed into the liver and incubated for 15-20 minutes at room temperature. The liver was cut into small pieces and kept on constant stirring for 20 minutes. It was then sieved through 40μ-mesh to remove connective tissue. Cell suspension was collected Hank's medium containing Ca²⁺ and Mg²⁺ salts was added and kept for gravity sedimentation. The supernatant containing RBCs and dead or broken cells was removed. The cell suspension was washed 4-5 times with Hanks medium, till hepatocyte suspension was obtained. The cells were then suspended in Dulbecco's Modified Eagles Medium (DMEM). The cell yield and viability were checked and the cells were stored at 4° C.

In accordance with this invention the hepatocytes are isolated and characterized.

Isolation of Hepatocytes:

The animals were sacrificed and livers were collected in cold and sterile conditions. They were processed for the isolation of hepatocytes in a laminar flow unit under aseptic conditions. The liver was perfused extensively with Hank's buffer through the portal vein, using a polythene catheter, until it became colourless. Collagenase solution was passé into the liver and incubated at room temperature. The liver was cut into small pieces and kept on constant stirring for 20 minutes. It was then sieved to remove connective tissue. Cell suspension was collected; Hank's medium containing Ca²⁺ and Mg²⁺ salts was added and kept for gravity sedimentation. The supernatant containing RBCs and dead or broken cells was removed. The cell suspension was washed 4-5 times with Hanks medium, till hepatocyte suspension was obtained. The cells were then suspended in a medium. The cell yield and viability were checked and the cells were stored at 4° C.

Human Fetus

Isolation of hepatocytes was carried out in a laminar flow unit under aseptic conditions as described by Habibullah et at 1990. The fetus was placed in a supine position after thoroughly cleaning the chest and abdomen with ethanol. The chest was opened from the 9^{th} costochondral joint on one side to the other. Longitudinal incisions were given on both sides along the costochondral junction to reach the level of the manubrium sterni. A thin polythene catheter of 22-24 gauge, was introduced into the umbilical cord vein and passed down for about 3-5 cms, and the cord was ligated to the catheter. Initially 10-30 ml of Hank's buffer was perfused until the liver swelled. The pericardium was cut and an incision was given to the inferior vena cava and the perfusate was allowed to flow out through the opening. The liver was perfused until it became colourless and it was gently pressed to remove the excess buffer. The inferior vena cava was clamped and collagenase solution (5-10 ml) was passed into the liver, and incubated for 10-15 min at room temperature. The liver was separated from the body and freed of connective and vascular tissue with the help of scalpel blade. Weight of the liver was taken and it was cut in small pieces and kept on constant stirring for 20 minutes. It was then sieved through 40 microns mesh to remove connective tissue, the isolated cell suspension was collected in a 250 ml conical flask, and Hank's medium containing Ca²⁺ and Mg²⁺salts was added and kept for gravity sedimentation. The supernatant containing RBCs and dead or broken cells was aspirated. The cell suspension was washed 4-5 times with Hank's medium till a pure hepatocyte suspension was obtained. The cells were then suspended in Dulbecco's Modified Eagles medium (DMEM). The cell yield/gm tissue was calculated, viability of the hepatocytes was checked and they were stored at 4° C.

Morphology:

Haematoxylin and Eosin (H&E) Staining:

Principle:

Haematoxylin is a base with negative charge and binds positive charged particles like. RNA in cytoplasm, and DNA in the nucleus, which are strained purple. Eosin is a positive charged dye and binds with most of cytoplasmic element giving cytoplasm a pink colour.

Reagents:

Haematoxylin solution: Haematoxylin solution (Harris), Nova Biotech.

Eosin solution: Eosin yellow stain solution, 2% w/v, Nice Chemicals Pvt. Ltd.

Fixation:

Cell suspension at a concentration of 0.1×10⁶ cells/ml was subjected to cytospin (Shandon Cytospin, Shandon Southern Products Ltd., Cheshire, UK) at 1000 rpm×10 min. Slides so prepared were immediately fixed in ethanol and acetone (1:1) at room temperature. Slides were stained with haematoxylin and eosin (H&E).

Autopsy samples were fixed in 10% formaldehyde. They were dehydrated and put in xylene. Then, rectangular blocks of paraffin were made with the tissue and thin paraffin sections were cut on slides and they were stained with haematoxylin and eosin (H&E).

Procedure (H & E Staining):

The slides were first stained with haematoxylin solution for 4-5 minutes. They were washed in running tap water for 3 minutes. They were decolourised in 95% alcohol by giving 2-3 dips. They were kept on blotting paper and then stained with eosin solution for 2 minutes. They were given two changes of 95% alcohol for 1-2 minutes each. The slides were given two changes of acetone 3 minutes each followed by two changes of xylol 3 minutes each. Finally the slides were mounted in D.P.X and viewed under microscope.

Staining:

They were decolourised in alcohol. They were kept on blotting paper and then stained. They were given two changes of the slides each followed by two changes of xylol minutes each.

Characterization:

The percentage of viable cells was calculated as the number of cells unstained divided by total number of cells (stained+unstained cells)×100.

MTT Assay:

MT assay was done by the method of Mossman 1983 with slight modification. Cells were taken and centrifuged. To the cell pellet, MTt reagent was added and incubated. The cell suspension was centrifuged and to the cell pellet, isopropanol was added and kept at room temperature. The purplish blue colour of the supernatant was read at 540 nm. The amount of formazan formed was expressed as μM formazan/10⁶ cells.

Membrane Integrity:

Hepatocyte suspension was centrifuged and the supernatant was separated. The pellet was solubilized by adding solubilizing solution as described by Mamprin et al. (1995). The LDH activity was determined in the pellet and in the supernatant by using a commercial LDH kit (E. Merck India Ltd.). To reaction solution taken in different curvets sample (supernatant, cell lysate) were added. They were mixed, incubated for 1 minute and absorbance was read at 340 nm. The change in absorbance was measured every minute. Percentage of LDH leakage was calculated as the ratio of percentage LDH activity in the supernatant to the total percentage LDH activity in supernatant to the total percentage LDH activity in supernatant and cell pellet.

Na⁺K⁺ ATPase Activity:

Na⁺K⁺ ATPase activity was estimated by the method of Sawas and Gilbert (1981). The hepatocytes were homogenized in sucrose using glass-teflon homogeniser and the Na⁺K⁺ ATPase was estimated in the final reaction mixture of (1) Nacl, KCl, MgCl₂, EDTA. Imidazole and oubain at pH 7.4. The assay was carried out with protein. The reaction was started by adding ATP and incubating minutes. The reaction was terminated and the inorganic phosphate (Pi) was determined from the supernatant. Enzyme activity was calculated as the difference in the Pi content between the total ATPase and Mg²⁺ ATPase activity.

Estimation of Phosphorous:

Phosphorus was estimated by the method as described by Raghuramulu at al. (1983). To test sample, reagent C was added, mixed and incubated. This was allowed to attain room temperature and the absorbance was read against the blank. A calibration curve of phosphorus was done and the activity of Na⁺K⁺ATPase was expressed as μM Pi/hr/mg protein.

Lipid Peroxidation:

Malondialdehyde was estimated by the method of Ohkawa et al. (1979), with slight modifications. Cell suspension (5×10⁶ cells) was added to Sodium dodecyl sulphate, acetic acid and the pH of the solution was adjusted with NaOH. To this aqueous solution of 2-thiobarbituric acid was added. The volume was made up to 4 ml with distilled water and then heated in boiling water bath for 60 minutes. The tubes were capped with marbles to prevent condensation. After cooling under tap water, 1 ml of distilled water and the mixture of n-butanol and pyridine (15:1) was added and shaken vigorously. The tubes were then centrifuged and the organic layer was aspirated out, and its absorbance at 532 nm was measured in a colorimeter. The levels of lipid peroxides were expressed as n moles of MDA formed/mg of protein.

Detoxification:

Cells were incubated with ammonium chloride. At the end of 1 hour, supernatant was collected and the amount of urea formed was estimated by a commercial kit (E.Merck India Limited). To 1 ml of reaction solution taken in different tubes (blank, standard, test), distilled water, urea standard, and sample were added respectively. They were mixed, and incubated. Absorbance. (A1) was measured at 340 nm. They were again incubated exactly after 60 seconds, and absorbance (A2) was measured separately for each tube at 340 nm, and the amount of urea formed was calculated and expressed as mM urea/10⁶ cells.

Cytochrome P450 Activity:

Cytochrome P450 activity was measured using diazepam as substrate by HPLC method. The HPLC system consisted of Waters LC Module-1 (Water's Milford, Mass., USA) and the detector used as photodiode array detector (PDA). Each incubation mixture in a total volume of 0.25 ml contained 0.125 ml potassium phosphate buffer (pH 7.4), 0.1 ml (4×10⁶) hepatocytes, Diazepam and NADPH. Reaction was terminated with methanol. Incubation mixture without Diazepam served as blank. For the purpose of estimation of Diazepam in samples, a similar incubation mixture spiked with Diazepam was terminated immediately which served as standard. After terminating the reaction and centrifuging, Organic phase (1 ml) was separated and evaporated to dryness under nitrogen. The residue was reconstituted with mobile phase and was injected into HPLC. The mobile phase was pumped through the stationary phase at a flow rate of 1 ml/min. The eluent was monitored for metabolites (Oxazepam and desmethyl diazepam) and drug (diazepam) using a PDA detector operating at 247 nm. Under these conditions, the retention times for metabolites (Oxazepam and desmethyl diazepam) and drug. The activity of the enzyme was expressed as the percentage of diazepam disappeared.

Glutathione-S-Transferase (GST) Activity:

GST activity was measured by the method of Habig et al. (1974). To 1.0 ml of phosphate buffer, 0.1 ml CDNB and 0.1 ml of cytosolic fraction were added. The volume was adjusted with distilled water. The reaction mixture was pre-incubated. The reaction was started by the addition of 0.1 ml of glutathione solution and the absorbance was followed at 340 nm (Beckman DU 640 B sprectrophotometer). The reaction mixture without cytosolic fraction was used as a blank. Activity of the enzyme was expressed as μmol CDNB-GSH conjugate/mg protein.

Estimation of Protein:

Protein was estimated by the method of Lowry et al. (1951). To 100 μl of test sample, distilled water was added. 5 ml of solution (alkaline copper sulphate reagent) was added, mixed and kept at room temperature. To this diluted Folin reagent was added, mixed well and incubated at room temperature for colour development. The absorbance was read at 680 nm. BSA calibration curve was done using 10, 20, 30, 40, 60, 80, 100 μg of protein. The final concentration of protein was expressed as μg/ml of cell lysate.

Viability:

Trypan Blue Dye Exclusion Test (TBE):

Principle:

The Test is Based on the Exclusion of TBE by Viable Cells, whereas Dead Cells are Stained by this Dye.

Reagents:

Phosphate buffered saline (PBS): 8.0 gm of NaCl was dissolved in 800 ml of double distilled water. To this 1.21 gm of K₂HPO₄ and 0.34 gm of KH₂PO₄ were added, pH was adjusted to 7.3 and the volume was made up to 1 litre.

Trypan blue solution: 0.4% trypan blue solution in PBS.

Procedure:

The staining of hepatocytes was done by the original method of Jauregui et al. (1981). Cell suspension was diluted 1:1 with trypan blue solution and counted in a haemocytometer under the microscope. The percentage of viable cells was calculated as the number of cells unstained divided by total number of cells (stained+unstained cells)×100.

MTT Assay:

Principle:

The reaction involves the conversion of tetrazolium salt (MTT), a pale yellow substrate, by dehydrogenase enzymes in mitochondria to formazan, which is a purplish blue compound. This is dissolved in isopropanol and absorbance in measured spectrophotometrically at 540 nm.

Reagents:

MTT reagent 100 mg of MTT (3-[4,5-dimethylthiozol-2yl]-2,5-diphenyl tetrazolium bromide, Sigma Chemical Co.) was dissolved in 100 ml of Hank's medium, pH 7.4.

Isopropanol (Analytical Grade)

Procedure:

MTT assay was done by the method of Mossman 1983 with slight modification. Cells were taken at a concentration of 4×10⁶ cells/ml, and centrifuged at 1000 rpm. To the cell pellet, 3 ml of MTT reagent was added and incubated at 37° C. for 2 hours. The cell suspension was centrifuged and to the cell pellet, 1 ml of isopropanol was added and kept at room temperature for 20 minutes. The purplish blue colour of the supernatant was read at 540 nm. The amount of formazan formed was expressed as μM formazan/10⁶ cells.

Membrane Integrity:

LDH Leakage

Principle:

The activity of LDH is measured by a reaction that involves the conversion of pyruvate to lactate using NADH as substrate.

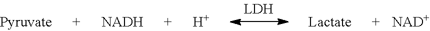

Reagents:

Solubilizing solution: 900 mg of NaCl was added to 80 ml of distilled water. 100 mg of BSA and 100 μl of Triton X 100 were added to the above solution and the volume was made up to 100 ml.

Buffer solution: It consists of 50 mmol/L phosphate buffer containing 0.6 mmol/L sodium pyruvate.

NADH solution: It contains 0.18 mmol/L NADH

Reaction solution: It was prepared by mixing 4 parts of buffer solution with 1 part of NADH solution.

Procedure:

Hepatocyte suspension was centrifuged at 700×g for 10 minutes and the supernatant was separated. The pellet was solubilized by adding 200 μl of solubilizing solution as described by Mamprin et al. (1995). The LDH activity was determined in the pellet and in the supernatant by using a commercial LDH kit (E.Merck India Ltd.).

To 500 μl of reaction solution taken in different cuvets, 10 μl of sample (supernatant, cell lysate) were added. They were mixed, incubated for 1 minute and absorbance was read at 340 nm. The change in absorbance was measured every minute for 3 minutes. Percentage of LDH leakage was calculated as the ratio of percentage LDH activity in the supernatant to the total percentage LDH activity in supernatant and cell pellet

Na⁺K⁺ATPase Activity:

Principle:

The total ATPase activity is measured using ATP as substrate in the absence of oubain. The activity is the presence of oubain is taken as Mg²⁺dependent ATPase activity. The difference in activity with and without oubain gives Na⁺K⁺ATPase activity.

Reagents:

Sucrose (0.25 M): 605.7 mg of Tris base was dissolved in about 200 ml of double distilled water and the pH was adjusted to 7.4 with 1 N HCl and the volume was made up to 1000 ml. 8.56 gm of sucrose and 10.16 mg of MgCl₂ were dissolved in 60 ml of 5 mM Tris HCl (pH 7.4), and finally the volume was adjusted to 100 ml with Tris HCl.

Cocktail solution: It consists of 240 mM NaCl, 20 mM KCl, 10 mM MgCl₂, 2 mM EDTA and 1.7 mM Imidazole. It was prepared by adding 1.403 gms of NaCl, 0.149 gms of KCl, 0.203 gms of MgCl₂, 74.4 mg of EDTA and 1.16 gms of Imidazole to 60 ml of distilled water, pH was adjusted to 7.4 and the volume was made up to 100 ml by adding distilled water.

Stock ATP (40 Mm): 0.2205 gms of ATP was dissolved in 10 ml of distilled water. Working ATP (4 mM) was prepared by adding 1 ml of stock ATP to 9 ml of double distilled water.

Stock Oubain (10 mM): 72.86 mg of oubain was dissolved in 10 ml of distilled water. Working oubain solution was prepared by adding 1 ml of stock oubain to 9 ml of double distilled water.

Procedure:

Na⁺K⁺ATPase activity was estimated by the method of Sawas and Gilbert (1981). The hepatocytes (4×10⁶) were homogenized in 0.25% sucrose using glass-teflon homogeniser and the Na⁺K⁺ATPase was estimated in the final reaction mixture of (1) 240 μmoles NaCl, 20 μmoles MgCl₂, 2 μmoles EDTA, 1.7 μmoles Imidazole and 10 μmoles of oubain at pH 7.4. The assay was carried out with 100 μg of protein. The reaction was started by adding 4 μmoles of ATP and incubated at 37° C. for 30 minutes. The reaction was terminated with 1 ml of 10% TCA, and the inorganic phosphate (Pi) was determined from the supernatant. Enzyme activity was calculated as the difference in the Pi content between the total ATPase and Mg²⁺ATPase activity.

Estimation of Phosphorus:

Principle:

Phosphate reacts with ammonium molybdate to give rise to phosphomolybdate complex, which is reduced to a more stable colour complex with ascorbic acid.

Reagents:

Ascorbic add (10%): 10 gm of ascorbic acid was dissolved in 60 ml of double distilled water and volume was made up to 100 ml with the same.

Ammonium molybdate (2.5%): 2.5 gms of ammonium molybdate was dissolved in 60 ml of double distilled water and volume was made up to 100 ml.

Sulphuric acid (6N): 90 ml of distilled H₂O was taken and to this 18 ml of concentrated H₂SO₄ was added, slowly

Reagent C: It was prepared by mixing one volume of 6N H₂SO₄, two volumes of distilled water, one volume of 2.5% ammonium molybdate and one volume of ascorbic acid. It was prepared freshly.

Phosphorus standard: 35.1 mg of KH₂PO₄ was dissolved in water and transferred to a 100 ml volumetric flask 1 ml of 10 N H₂SO₄ was added and the solution was made up to 100 ml with water and mixed. This solution contained 0.4 mg of phosphorus in 5 ml.

Procedure:

Phosphorus was estimated by the method as described by Raghuramulu et al. (1983). To 100 μl of test sample 4 ml of reagent C was added mixed and incubated at 37° C. for 1.5-2 hours. This was allowed to attain room temperature and the absorbance was read at 820 nm against the blank. A calibration curve of phosphorus was done and the activity of Na⁺K⁺ATPase was expressed as μM Pi/hr/mg protein.

Lipid Peroxidation:

Principle:

Malondialdehyde is the end product of lipid peroxidation of polyunsaturated fatty acids. MDA was estimated by utilizing its property to react with 2-thiobarbituric acid (TBA). One mole of MDA reacts with 2 moles of TBA to form a pink coloured condensation product, a trimethane that is spectrophotometrically measured at 532 nm.

Reagents

Sodium dodecyl sulphate (SDS) 8.1%: It was prepared by adding 810 mg of SDS to 6 ml of double distilled water and the total volume was made 10 ml.

2-thiobarbituric acid in aqueous solution (0.8%): It was prepared by adding 80 mg of TBA to 10 ml of double distilled water which was dissolved on warming the solution.

Glacial acetic acid (20.0%)

Sodium hydroxide solution to adjust pH 3.5 (5.0%)

Sodium chloride (0.9%)

Potassium chloride (1.15%)

n-butanol (Analytical grade)

Pyridine (Analytical grade)

Procedure:

Malondialdehyde was estimated by the method of Ohkawa at al (1979), with slight modifications. 0.2 ml of cell suspension (5×10⁶ cells) was added to 0.2 ml 8.1% SDS, 1.5 ml of 20% acetic acid and the solution was adjusted to pH 3.5 with 5% NaOH. To this 1.5 ml of 0.8% aqueous solution of TBA was added. The volume was made up to 4 ml with distilled water and then heated in boiling water bath for 60 minutes. The tubes were capped with marbles to prevent condensation. After cooling wider tap water, 1 ml of distilled water and 5 ml of the mixture of n-butanol and pyridine (15:1) was added and shaken vigorously. The tubes were then centrifuged at 3000 rpm for 15 minutes and the organic layer was aspirated out and its absorbance at 532 nm was measured in a colorimeter. The levels of lipid peroxides were expressed as n moles of MDA formed/mg of protein.

Detoxification:

Ammonia Loading Test and Urea Estimation:

Principle:

The detoxifying capacity of hepatocytes was measured by loading the hepatocytes with ammonium chloride and estimating the amount of urea formed spectrophotometrically. The reaction involved in the estimation of urea is as follows:

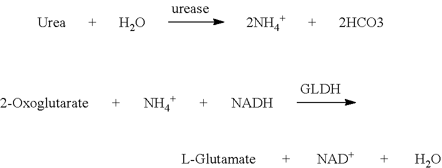

Reagent:

10 mM ammonium chloride: 534.9 mg of ammonium chloride was dissolved in 100 ml of Hank's medium.

Buffer solution: It consists of a Tris buffer 120 mmol/L pH 7.8 containing 7 mnol/L of 2-oxoglutarate, 0.6 mmol/L ADP, >6KU/L Urease and >1KU/L GLDH.

NADH solution: It contains 0.25 mmol/L of NADH.

Urea standard: It consists of 50 mg/dL of urea.

Reaction solution: 0.5 ml of NADH solution was added to 2 ml of buffer solution, mixed and left for 10 minutes at 25° C.

Procedure:

4-5×10⁶ cells were incubated with 10 mM ammonium chloride at 37° C. in 5% CO₂. At the end of 1 hour, 20 μl of supernatant was collected and the amount of urea formed was estimated by a commercial kit (E.Merck India Limited).

To 1 ml of reaction solution taken in different tubes (blank, standard, test), 10 μl of distilled water, 10 μl of urea standard, and 14 μl of the sample were added respectively. They were mixed, and incubated for 60 seconds at 30° C. Absorbance (A1) was measured at 340 nm. They were again incubated exactly after 60 seconds, and absorbance (A2) was measured separately for each tube at 340 nm, and the amount of urea formed was calculated and expressed as mM urea/10⁶ cells.

Cytochrome P450 Activity:

Principle:

The measurement of Cytochrome P450 activity was based on the metabolism of diazepam as substrate by HPLC method. Monitoring the eluent for metabolites (Oxazepam and desmethyl diazepam) and drug (diazepam) using a PDA detector operating at 247 nm shows the amount of diazepam metabolized which is directly related to the cytochrome P450 activity.

3.9.2.2 Reagents:

Potassium dihydrogen (KH₂PO₄0.2M) solution: It was prepared by adding 27.2 gms of KH₂PO₄ in 60 ml of double distilled water and the volume was made up to 100 ml.

Sodium hydroxide (NaOH, 0.2M) solution: 8.0 gms of NaOH was added to 60 ml of distilled water and volume was made up to 100 ml with the same.

Potassium phosphate buffer: (0.2M, pH 7.4): It was prepared by adding 50 ml of 0.2M KH2PO4 to 39.1 ml of 0.2M NaOH and the volume was made up to 100 ml with double distilled water.

Diazepam (7 mM): It was prepared by dissolving 19.88 mg of diazepam in 10 ml of methanol

Nicotinamide adenine dinucleotide phosphate reduced (NADPH): 100 mg of NADPH was dissolved in 2.5 ml of double distilled water.

Mobile phase: It consisted of methanol: acetonitrile: water in the ratio of 35:20:45(v/v);

Stationary phase: It was a Hichrom-HIRB C18 column (4.6×250 mm, 5 □M)

Procedure:

Cytochrome P450 activity was measured using diazepam as substrate by HPLC method. The HPLC system consisted of Water LC Module-1 (Water's Milford, Mass., USA) and the detector used was photodiode array detector (PDA).

Each incubation mixture in a total volume of 0.25 ml contained 0.125 ml potassium phosphate buffer (pH 7.4), 0.1 ml (4×10⁶) hepatocytes. 70 μm Diazepam and 20 μl of (40 mg/ml) NADPH. Reaction was terminated with 1 ml of methanol. Incubation mixture without Diazepam served as blank. For the purpose of estimation of Diazepam in samples, a similar incubation mixture spiked with Diazepam (70 μM) was terminated immediately which served as standard. After terminating the reaction and centrifuging at 1000 rpm for 20 minutes, Organic phase (1 ml) was separated and evaporated to dryness under nitrogen at 40° C. The residue was reconstituted with 200 μl of mobile phase and 50 μl was injected into HPLC. The mobile phase was pumped through the stationary phase at a flow rate of 1 ml/min. The eluent was monitored for metabolites (Oxazepam and desmethyl diazepam) and drug (diazepam) using a PDA detector operating at 247 nm. Under these conditions, the retention times for metabolites (Oxazepam and desmethyl diazepam) and drug were 18.8, 12.8 and 14.9 minutes. The activity of the enzyme was expressed as the percentage of diazepam disappeared.

Glutathione-S-Transferase (GST) Activity:

Principle:

Activity of the enzyme was measured by following the increase in absorbance at 340 nm using 1-chloro-2,4-dinitrobenzene as the substrate. Decrease in absorbance of substrate when conjugated with glutathione forms the basis of spectrophotometric measurement

3.93.2 Reagents:

Sucrose (0.25 M): 605.7 mg of Tris base was dissolved in about 200 ml of double distilled water and the pH was adjusted to 7.4 with 1 N HCl and the volume was made up to 1000 ml. 8.56 gm of sucrose and 10.16 mg of MgCl₂ were dissolved in 60 ml of 5 mM Tris HCl (pH 7.4) and finally the volume was adjusted to 100 ml with Tris HCl.

Solution A (0.3 M NaH₂PO₄): 4.68 gms of NaH₂PO₄ was dissolved in 100 ml of double distilled water.

Solution B (0.3 M Na₂HPO₄): 4.23 gms of Na₂HPO₄ was dissolved in 100 ml of double distilled water.

Phosphate buffer (0.3M): It was prepared by mixing 50 ml of solution A with 26 ml of solution B pH was adjusted to 6.5 and the volume was made to 100 ml with double distilled water.

Glutathione solution (30 mM): 23 mg of glutathione was dissolved in 2.5 ml of 0.3M phosphate buffer pH6.5 to give the final concentration of 30 mM glutathione.

1 chloro 2,4-dinitro benzene (CDNB) solution: 15.2 mg of CDNB was dissolved in 2.5 ml of 95% ethanol.

Preparation of Cytosol: The cells were homogenized in 0.25% sucrose using glass-teflon homogenizer. The homogenate was centrifuged at 700 g for 15 minutes for the removal of nuclear material and the cell debris. The supernatant was centrifuged at 15,00,000 g for 60 minutes to get the clear cytosolic fraction and this was aspirated and used for enzyme assay.

Procedure:

GST activity was measured by the method of Habig et al. (1974). To 1.0 ml of phosphate buffer, 0.1 ml CDNB and 0.1 ml of cytosolic fraction were added. The volume was adjusted to 2.9 ml with distilled water. The reaction mixture was pre-incubated at 37° for 5 min. The reaction was started by the addition of 0.1 ml of glutathione solution and the absorbance was followed for 5 min at 340 nm (Beckman DU 640 B spectrophotometer). The reaction mixture without cytosolic fraction was used as a blank. Activity of the enzyme was expressed as μmol CDNB-GSH conjugate/mg protein.

Estimation of Protein:

Principle:

Proteins react with Folin-phenol reagent to form a coloured complex which can be measured colorimetrically. The final colour developed in the reaction is a result of (1) biuret reaction of protein with copper in an alkaline medium, and (2) reduction of the phosphomolybdic acid to phosphotungstic acid reaction by the tyrosine and tryptophan residues of the treated proteins.

Reagents:

Sodium hydroxide solution (0.1 N): 2 gm of NaOH was dissolved in double distilled water and the volume was made up to 500 ml.

Solution A: (2% sodium carbonate): 2 gm of Na CO3 was dissolved in 0.1 N NaOH and finally the volume was made up to 100 ml.

Solution B (0.5% copper Sulphate): 500 mg of CuSO₄5H₂O (SD Fine Chem. India) was dissolved in 70 ml of double distilled water and the volume was made up to 100 ml.

Solution C (1% potassium sodium tartarate): 1 gm of potassium sodium tartarate (BDH Laboratories, India) was dissolved in 70 ml double distilled water and the volume was made to 100 ml.

Solution D (alkaline copper sulphate reagent): To 49 ml of solution A, 0.5 ml of solution B and 1 ml of solution C was added.

Form's Reagent (2N): The working Folin's reagent was prepared by mixing 1 part of Folin's reagent with 1 part of double distilled water just before use.

Standard protein solution: Bovine serum albumin (Sigma Chemical Co. MO. USA) was used for the preparation of a standard protein solution. A standard solution containing 200 μg of protein per ml was prepared by dissolving 10 mg of BSA in 50 ml double distilled water.

Procedure:

Protein was estimated by the method of Lowry at al. (1951). To 100 μl of test sample, 0.9 ml of distilled water was added 5 ml of solution D (alkaline copper sulphate reagent) was added, mixed and kept for 15 minutes at room temperature. To this 0.5 ml of diluted Folin reagent was added mixed well and incubated for 30 minutes at room temperature for colour development. The absorbance was read at 680 nm. BSA calibration curve was done using 10, 20, 30, 40, 60, 80, 100 μg of protein. The final concentration of protein was expressed as μg/ml of cell lysate.

Invitro Comparison of Cytotoxicity of Human Serum with Goat, Human Fetal and Pig Hepatocytes Using MTT Assay:

Human AB serum was obtained from 10 healthy persons. AB serum was used for the study to exclude the effect of blood group antibodies. As a control, the human AB serum was subjected to inactivation of complement by heating the serum at 56° C. for 60 minutes. The concentration of serum used for the incubation with the hepatocytes was made 80% with DMEM (pH 7.4).

Hepatocytes isolated from goat, human fetus and pig at a concentration of 1.0×10⁶ cells/ml were incubated with 100 μl each of both normal and heat inactivated human sera separately for a period 2 hours at 37° C. They were then centrifuged at 700×g for 5 minutes. The pellet consisting of hepatocytes was collected and subjected to MTT assay as described in section 3.7.2.

Effect of Antibody Depleted Human Serum on Goat and Pig Hepatocytes:

Preparation of Rabbit Red Blood Cells (Fixed):

Rabbit blood was collected from ear vein in EDTA and RBCs were separated using Ficoll hypaque. They were washed and resuspended in 20-30% saline. Glutaraldehye was added to a final concentration of 0.5% and incubated overnight. Than they were washed three times and resuspended in 100 mM glycine and incubated overnight. They were washed and resuspended in 1% BSA and kept in cold.

Absorption of Anti-Gal Antibody from Human Serum:

To 0.5 ml of packed rabbit RBCs, 1.0 ml of normal human serum was added and incubated for two hours at 4° C. with occasional mixing. Serum was then centrifuged to remove rabbit RBCs.

Incubation of Goat and Pig Hepatocytes with Normal as well as Antibody Depleted Human Serum:

Hepatocytes (1×10⁶) were incubated with 100 μl of 80% normal as well as antibody depleted human serum separately. 10 μl of rabbit serum (complement source) was added and incubated at 37° C. for 2 hours. The viability of hepatocytes was checked by MTT assay.

Invivo Study:

Animals:

Wistar rats (225-250 g) were obtained from the National Institute of Nutrition, Hyderabad India. They were housed in individual wire bottomed cages in air-conditioned rooms (22±1° C.). All the animals had free access to standard feed and water ad libitum. They were maintained in 12 hours dark and light cycle.

All the experiments with animals were carried out by strictly following the guidelines issued by the Committee for the Purpose of Control and Supervision of Experiments on Animals, Ministry of Social Justice and Empowerment, Government of India. In our experimental protocol goats served as the source of hepatocyted, where as rats served as recipients. The total number of animals (rats) included in the study was sixty four. They were divided into four experimental groups. Group I, which served as control received only normal saline. Group II received only D-galactosamine, Group III received non-irradiated hepatocytes after D-galactosamine induced lever injury and Group IV received UV-B irradiated hepatocytes (1250 Joules/m²) after D-galactosamine induced liver injury.

Induction of Liver Failure in Wistar Rats Using D Galactosamine:

Liver failure was induced by injection of D-galactosamine hydrochloride 875 mg/Kg body weight prepared in 5% Dextrose. Galactosamine was injected intra-peritoneally at the rate of 0.5 ml/min.

Follow-Up of the Animals:

After injection of D-galactosamine, the animals were kept under strict observation. They were fed glucose water and all the clinical signs and symptoms especially neural reflex were observed. It was found that animals were going into coma within 24-36 hours of D-galactosamine injection.

Transplantation of Hepatocytes:

Transplantation of hepatocytes was done after 24 hr of D-galactosamine hydrochloride injection after checking their prothrombin time. When the prothrombin time was reaching one minute, immediately hepatocyte suspensions (in normal saline) were injected in group III animals through the infra-peritoneal route with a 20 gauge needle at a dose of 60×10⁶ cells/Kg body weight. Group I animals were injected with equal volume of normal saline. The following parameters were studied in all the groups.

Liver Function Tests:

Estimation of Total Bilirubin:

Principle:

Bilirubin reacts with diazotized sulphanillic acid (DSA) to form a red azo dye. The absorbance of this dye at 546 nm is proportional to the bilirubin concentration in the sample. Water-soluble bilirubin glucuronides reacts directly with DSA whereas the albumin-conjugated indirect bilirubin will only react with DSA in the presence of an accelerator.

Total bilirubin=Direct+Indirect Bilirubin

Sulphanillic acid+Sodium nitrite→DSA diazotized sulphanillic acid

Bilirubin+DSA+caffeine (accelerator)→Total Azobilirubin.

Reagents:

Reagent I (Sulphanillic acid Reagent): It consists of 14 mM sulphanillic acid, 0.2 M caffeine, and 0.42 M sodium benzoate in 0.25 M HCL

Reagent 2 (Nitrite Reagent): It consists of 14 mM sodium nitrite.

Procedure:

Total Bilirubin was determined using commercial kit (Wipro Biomed India). To 1.0 ml of reagent 1 and 40 μl of reagent 2, taken in a test tube, 100 μl of serum was added and mixed thoroughly. In the sample blank 1.0 ml of reagent 1 and 100 μl of serum were taken and reagent 2 was excluded. The tubes were mixed and kept for incubation at room temperature for 30 minutes. The absorbance of the sample was measured against the blank at 546 nm. Total bilirubin was expressed as mg/dL.

Estimation of Alanine Aminotransferase (ALT):

Principle:

ALT catalyses the reaction between L-Alanine and 2-Oxoglutarate (α KGA) and the pyruvate formed is reduced by NADH in a reaction catalysed by LDH. After the initial lag phase, the equilibrium of reaction (b) is far to the right. Hence the decrease in absorbance due to NADH oxidation is stoichiometric with ALT concentration. ALT

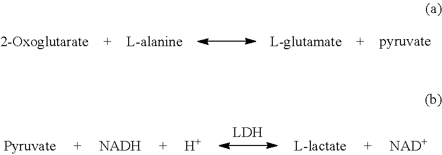

Reagents:

Tris buffer (100 mmol/L): It is a his buffer (100 mmol/L, pH 7.4) containing 500 mmol/L L-alanine, 1200 U/L LDH and 0.18 mmol/L NADH.

Substrate solution: It contains 15 mmol/L 2=oxoglutarate.

Reaction mixture: It is prepared by mixing 1 vial of reagent 2 into 1 bottle of reagent 1.

Procedure:

ALT was determined using commercial kit (Wipro Biomed, India). In a cuvette, 1.0 ml of reaction mixture was taken and 100 μl of serum was added, mixed and the absorbance was read at 340 nm after 1-minute incubation at room temperate. Again the absorbance was read after 1, 2 and 3 minutes. ALT activity was calculated and expressed as IU/L.

Estimation of Albumin:

Principle:

Bromocresol green in citrate buffer forms a coloured complex with albumin. The absorbance of this complex is proportional to the albumin concentration in the sample.

Reagents:

Colour reagent: It consists of 0.15 mM bromocresol green in 7.5 mM citrate buffer. PH 4.2.

Albumin standard: It contains 3 g/dL albumin.

Procedure:

Albumin was determined using commercial kit (Wipro Biomed India). 1.0 ml of colour reagent was taken in different test tubes (test, standard and blank). 10 μl of serum was added to the test, 10 μl of albumin standard was added to the standard tube and the blank tube consisted of only colour reagent. The tubes were mixed and incubated for 5 minutes at 20-25° C. The absorbance of the sample and the standard were measured at 520 nm against the reagent blank. The concentration of Albumin was expressed as g/dL.

Measurement of Prothrombin Time (PT):

Principle:

Thromboplastin in the presence of calcium activates the extrinsic pathway of blood coagulation mechanism. On addition of Liquiplastin reagent to normal anti-coagulated plasma the clotting mechanism is initiated forming a solid gel clot within a specified period of time.

Reagent:

Liquiplastin: It is a Calcium Thromboplastin Reagent, which is derived from rabbit brain.

Procedure:

PT was measured by a commercially available calcium reagent kit (LIQUIPLASTIN TULIP Diagnostics (P) Ltd, India). 0.1 ml of plasma was taken in a test tube and placed in a water bath for 3-4 minutes at 37° C. Then to it 0.2 ml of thromboplastin reagent (pre-warmed at 37° C.) was added and time taken for clot formation was estimated with the help of a stopwatch. The time recorded (in seconds) gave the prothrombin time.

Estimation of Blood Ammonia

Principle:

The method for the estimation of ammonia is based on reductive amination of 2-oxoglutarate, using glutamate dehydrogenase (GLDH) and NADPH as follows:

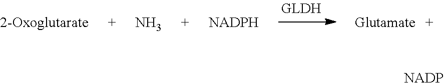

The decrease in absorbance at 340 nm due to oxidation of NADPH is proportional to the plasma ammonia concentration.

Reagents:

Reagent 1 (Ammonia assay solution): This reagent was reconstituted with 3.5 ml of de-ionized water. It consists of 3.4 mmol/L 2-Oxoglutarate and 0.23 mmol/L NADPH.

Reagent 2 (L-glutamate dehydrogenase solution): It consists of 1200 U/mlLGLDH and 50% v/v glycerol in phosphate buffer pH 7.4

Reagent 3 (Ammonia control solution): It contains 5 μg/ml ammonia as ammonium sulphate.

Procedure:

Ammonia was estimated using a commercial kit (Sigma Diagnostics, Sigma Chemical Company. USA). A series of cuvettes were set for Blank, Control and Test and 1.0 ml of Reagent 1 was taken in each cuvette. To Blank cuvette 0.1 ml of water to Control cuvette 0.1 ml Reagent 3 and to the test cuvette 0.1 ml of plasma was added. They were covered with parafilm and mixed by gentle inversion. The cuvettes were allowed to equilibrate for approximately 3 minutes at room temperature. Initial absorbance was read and recorded for each cuvette against water at 340 nm. Then 10 μl of reagent 2 was added to each cuvette mixed by gentle inversion and incubated for 5 minutes at room temperature. Final absorbance was read and recorded for each cuvette against water at 340 nm. The concentration of ammonia was calculated and expressed as μg ml.

Histopathological Study:

The autopsy samples of the liver were taken on different days post transplantation and haematoxylin eosin stained sections were prepared. The changes in the histology were studied.

Staining:

They were decolourised in alcohol. They were kept on blotting paper and then stained. They were given two changes of the slides each followed by two changes of xylol minutes each.

Characterization:

The percentage of viable cells was calculated as the number of cells unstained divided by total number of cells (stained+unstained cells))×100.

MTT Assay:

MT assay was done by the method of Mossman 1983 with slight modification. Cells were taken and centrifuged. To the cell pellet, MTt reagent was added and incubated. The cell suspension was centrifuged and to the cell pellet, isopropanol was added and kept at room temperature. The purplish blue colour of the supernatant was read at 540 nm. The amount of formazan formed was expressed as μM formazan/10⁶ cells.

Membrane Integrity:

Hepatocyte suspension was centrifuged and the supernatant was separated. The pellet was solubilized by adding solubilizing solution as described by Mamprin et al. (1995). The LDH activity was determined in the pellet and in the supernatant by using a commercial LDH kit (E. Merck India Ltd). To reaction solution taken in different curvets sample (supernatant, cell lysate) were added. They were mixed, incubated for a 1 minute and absorbance was read at 340 nm. The change in absorbance was measured every minute. Percentage of LDH leakage was calculated as the ratio of percentage LDH activity in the supernatant to the total percentage LDH activity in supernatant to the total percentage LDH activity in supernatant and cell pellet.

Na⁺K⁺ATPase Activity:

Na⁺K⁺ATPase activity was estimated by the method of Sawas and Gilbert (1981). The hepatocytes were homogenized in sucrose using glass-teflon homogenizer and the Na⁺K⁺ATPase was estimated in the final reaction mixture of (1) Nacl, KCl, MgCl₂ EDTA. Imidazole and oubain at pH 7.4. The assay was carried out with protein. The reaction was started by adding ATP and incubating minutes. The reaction was terminated and the inorganic phosphate (Pi) was determined from the supernatant. Enzyme activity was calculated as the difference in the Pi content between the total ATPase and Mg²⁺ ATPase activity.

Estimation of Phosphorous:

Phosphorus was estimated by the method as described by Raghuramulu et al. (1983). To test sample, reagent C was added, mixed and incubated. This was allowed to attain room temperature and the absorbance was read against the blank. A calibration curve of phosphorus was done and the activity of Na⁺K⁺ATPase was expressed as μM Pi/hr/mg protein.

Lipid Peroxidation:

Malondialdehyde was estimated by the method of Ohkawa et al (1979), with slight modifications. Cell suspension (5×10⁶ cells) was added to Sodium dodecyl sulphate acetic acid and the pH of the solution was adjusted with NaOH. To this aqueous solution of 2-thiobarbituric acid was added. The volume, was made up to 4 ml with distilled water and then heated in boiling water bath for 60 minutes. The tubes were capped with marbles to prevent condensation. After cooling under tap water, 1 ml of distilled water and the mixture of n-butanol and pyridine (15:1) was added and shaken vigorously. The tubes were then centrifuged and the organic layer was aspirated out and its absorbance at 532 nm was measured in a colorimeter. The levels of lipid peroxides were expressed as n moles of MDA formed/mg of protein.

Detoxification:

Cells were incubated with ammonium chloride. At the end of 1 hour, supernatant was collected and the amount of urea formed was estimated by a commercial kit (E.Merck India Limited). To 1 ml of reaction solution taken in different tubes (bionic, standard, test), distilled water, urea standard, and sample were added respectively. They were mixed and incubated. Absorbance (A1) was measured at 340 nm. They were again incubated exactly after 60 seconds, and absorbance (A2) was measured separately for each tube at 340 nm, and the amount of urea formed was calculated and expressed as mM urea/10⁶ cells.

Cytochrome P450 Activity:

Cytochrome P450 activity was measured using diazepam as substrate by HPLC method. The HPLC system consisted of Waters LC Module-1 (Water's Milford, Mass., USA) and the detector used as photodiode array detector (PDA). Each incubation mixture in a total volume of 0.25 ml contained 0.125 ml potassium phosphate buffer (pH 7.4), 0.1 ml (4×10⁶) hepatocytes, Diazepam and NADPH. Reaction was terminated with methanol. Incubation mixture without Diazepam served as blank. For the purpose of estimation of Diazepam in samples, a similar incubation mixture spiked with Diazepam was terminated immediately which served as standard. After terminating the reaction and centrifuging, Organic phase (1 ml) was separated and evaporated to dryness under nitrogen. The residue was reconstituted with mobile phase and was injected into HPLC. The mobile phase was pumped through the stationary phase at a flow rate of 1 ml/min. The eluent was monitored for metabolites (Oxazepam and desmethyl diazepam) and drug (diazepam) using a PDA detector operating at 247 nm. Under these conditions, the retention times for metabolites (Oxazepam and desmethyl diazepam) and drug. The activity of the enzyme was expressed as the percentage of diazepam disappeared.

Glutathione-S-Transferase (GST) Activity:

GST activity was measured by the method of Habig et al (1974). To 1.0 ml of phosphate buffer, 0.1. ml CDNB and 0.1 ml of cytosolic fraction were added. The volume was adjusted with distilled water. The reaction mixture was pre-incubated. The reaction was started by the addition of 0.1 ml of glutathione solution and the absorbance was followed at 340 nm (Beckman DU 640 B sprectrophotometer). The reaction mixture without cytosolic fraction was used as a blank. Activity of the enzyme was expressed as μmol CDNB-GSH conjugate/mg protein.

Estimation of Protein:

Protein was estimated by the method of Lowry et al (1951). To 100 μl of test sample, distilled water was added 5 ml of solution (alkaline copper sulphate reagent) was added, mixed and kept at room temperature. To this diluted Folin reagent was added, mixed well and incubated at room temperature for colour development. The absorbance was read at 680 mm. BSA calibration curve was done using 10, 20, 30, 40, 60, 80, 100 μg of protein. The final concentration of protein was expressed as μg/ml of cell lysate.

The present invention will be apparent from the accompanying drawings and the appended claims.

### BRIEF DESCRIPTION OF THE DRAWINGS

Isolation of Hepatocytes and Cell Yield:

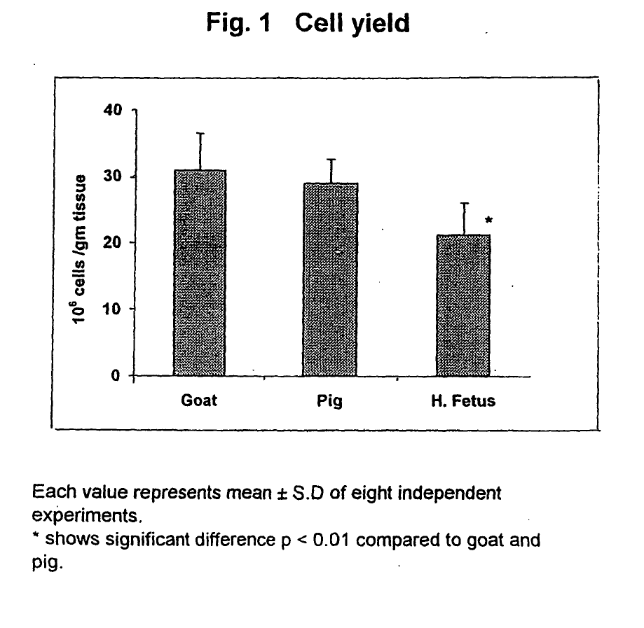

The isolation of hepatocytes by the two-step collagenase digestion method gave a cell yield of 21.2±4.8×10⁶ cells/gm wet tissue from human fetal liver. 30.9±5.6×10⁶ cells/gm wet tissue from goat liver and 29.0±3.6×10⁶ cells/gm wet tissue from pig liver (FIG. 1).

Invitro Comparative Study Goat, Pig and Human Fetal Hepatocytes:

Viability:

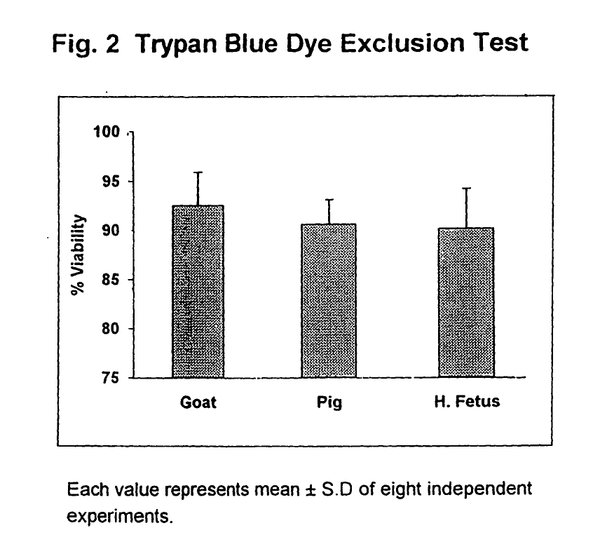

Trypan blue dye exclusion test (TBE): The viability as assessed by TBE was >90% in human fetal, goat and pig hepatocytes and comparable among the three groups (FIG. 2).

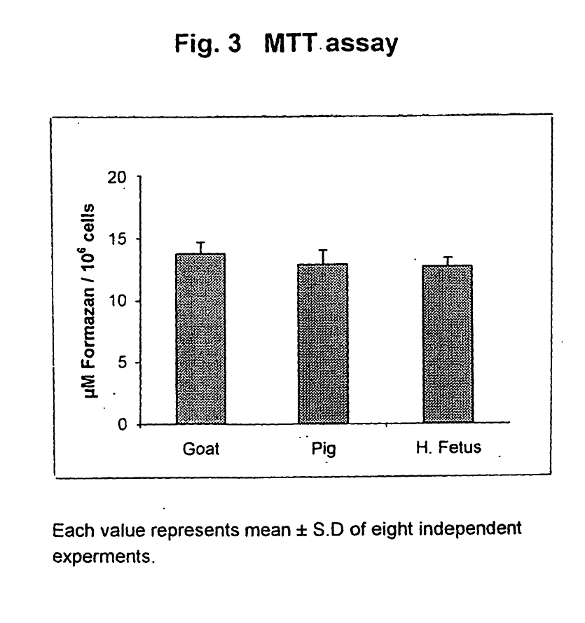

MTT assay: The viability as assessed by MTT assay was also comparable among the three groups (FIG. 3).

Membrane Integrity:

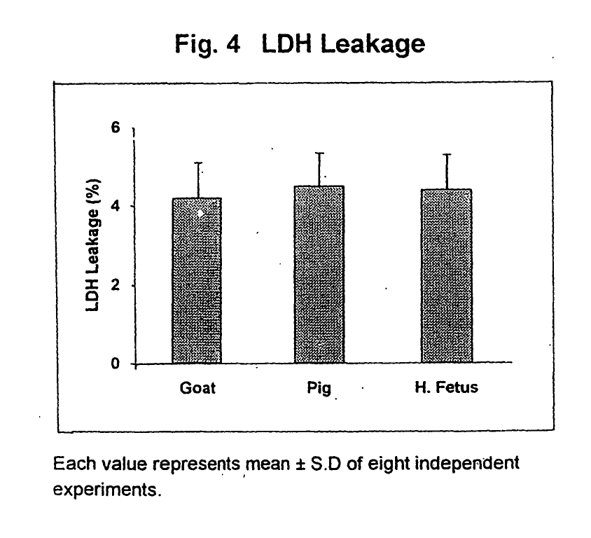

LDH leakage: The membrane integrity as assessed by percentage LDH leakage did not show significant difference among the three groups (FIG. 4).

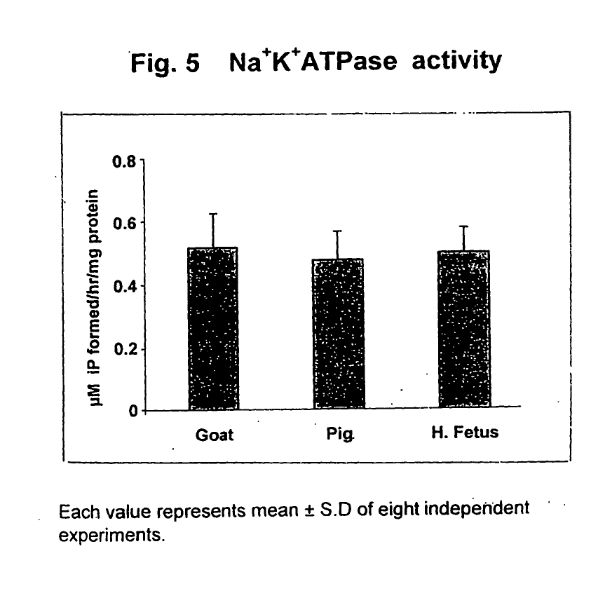

Na⁺K⁺ATPase activity: The activity of Na⁺K⁺ATPase was also comparable among the three groups (FIG. 5).

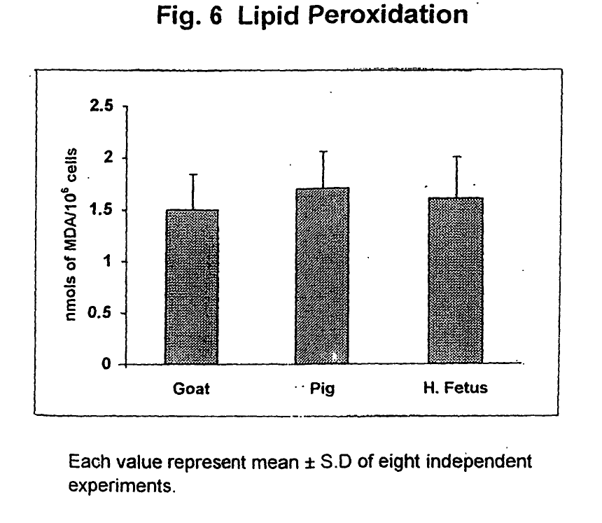

Lipid peroxidation: This was measured by the formation of malondialdehyde (MDA) and it was found that there was no statistically significant difference among the three groups (FIG. 6).

Detoxification

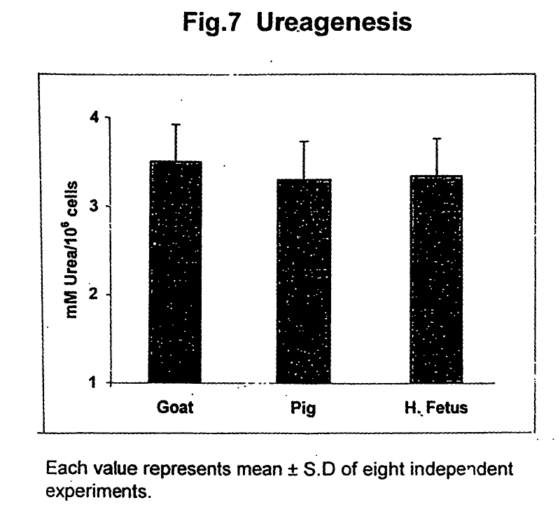

Ureagenesis: This was measured by the ammonia loading test and was found to be comparable among the three groups (FIG. 7).

Invitro Comparison of the Cytotoxicity of Human Serum with Goat, Pig and Human Fetal Hepatocytes:

Viability of the hepatocytes incubated with DMEM was considered as 100%. The viability of goat hepatocytes on incubation with heat inactivated human serum was 91.5±6.8% and on incubation with normal human serum it was found to be 8.9±6.7%, which was not statistically. Human fetal hepatocytes, on incubation with heat inactivated human serum showed availability of 92.8±5.5% and on incubation with normal human serum, their viability was found to be 93.5±5.4%. These values were comparable with the viability of goat hepatocytes under the same conditions. However, pig hepatocytes showed a statistically significant difference when they were incubated with heat inactivated and normal human sera. The values were found to be 88±6.5% and 65.51±8.5% respectively (p<0.01).

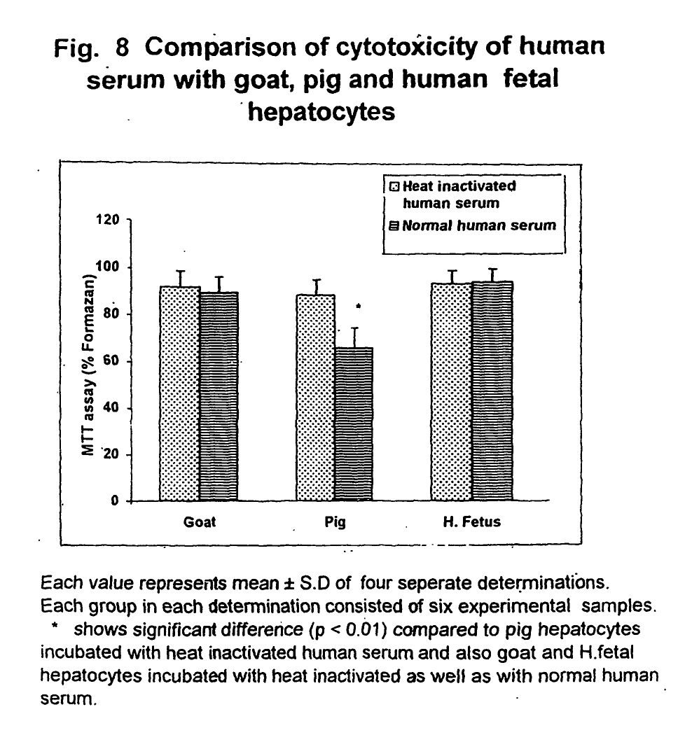

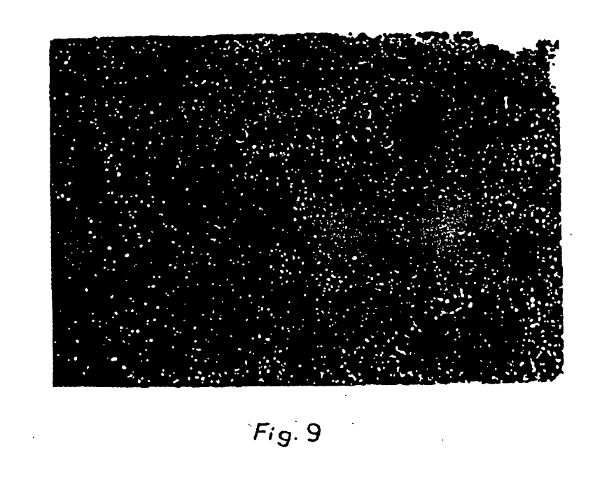

Pig hepatocytes on incubation with normal human serum showed a significant decrease (p<0.01) as compared to goat and human fetal hepatocytes incubated under the same conditions (FIG. 8).

Effect of Antibody-Depleted Human Serum on Goat and Pig Hepatocytes:

The viability of goat hepatocytes on incubation with antibody-depleted human serum was found to be 90.8±5.7% and their viability on incubation with normal human serum was 89.3±5.12% which were comparable. The viability of pig hepatocytes on incubation with antibody depleted human serum was 82±8.4% and their viability on incubation with normal serum was 65.5±8.6%, which was statistically significant (P<0.01). (Table 1).

Morphology of the Goat Hepatocytes (H&E Staining):

The morphology of hepatocytes was intact with cytoplasm stained in pink colour and distinct nucleus stained in blue colour (Photo 9).

Cytochrome P450 activity in goat hepatocytes: This was measured by invitro diazepam metabolism. (Table 2).

Invivo Study (Transplantation of Goat Hepatocytes in D-Galactosamine Induced FHF Rat Model):

Liver Function Parameters:

Prothrombin Time (PT):

This was significantly high after 24 hours of D-galactosamine liver injury (day 1), remained significantly high till day 5 (p<0.001), and reached the normal values on day 10 in group III animals. (Table 3).

Blood ammonia: The levels of Blood ammonia were found to be significantly high after 24 hours of D-galactosamine injection (p<0.001), which remained significantly high on day 5 (p<0.001) and reached normal value by day 10 in group III animals (Table 4).

Serum bilirubin: This level of Serum bilirubin was found to be highly significantly after 24 hours of D-galactosamine liver injury (p<0.001) and it remained statistically significant till day 10 (p<0.01) which then gradually decreased and reached the normal values by day 15 in group III animals (Table 5).

ALT activity: The Alanine aminotransferase activity was significantly high after 24 hours of D-galactosamine administration (P<0.001) remained highly significant on day 5 and day 10 (p<0.001) was still statistically significant till day 15 (p<0.01) but decreased and came back to normal on day 20 post transplantation in group III (Table 6).

Serum albumin: The Serum albumin levels did not show any significant difference among all the groups (Table 7).

Histopathology of Liver:

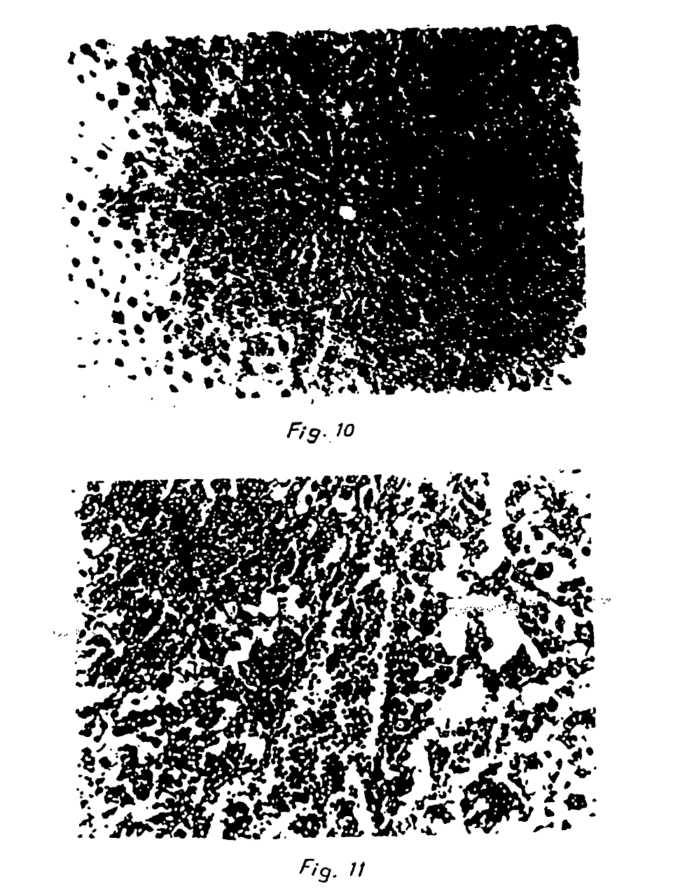

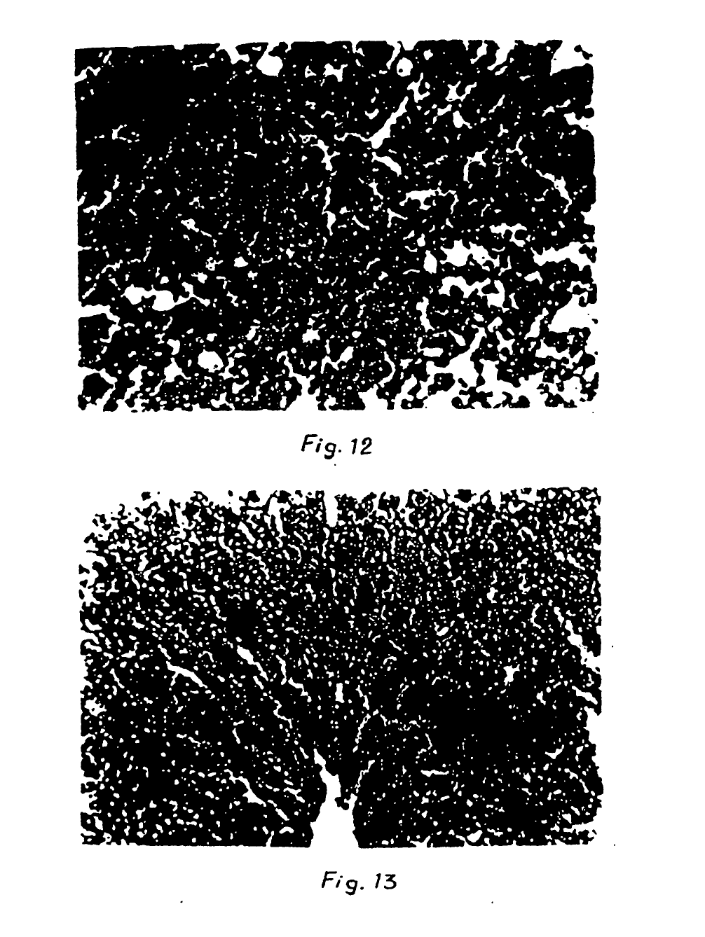

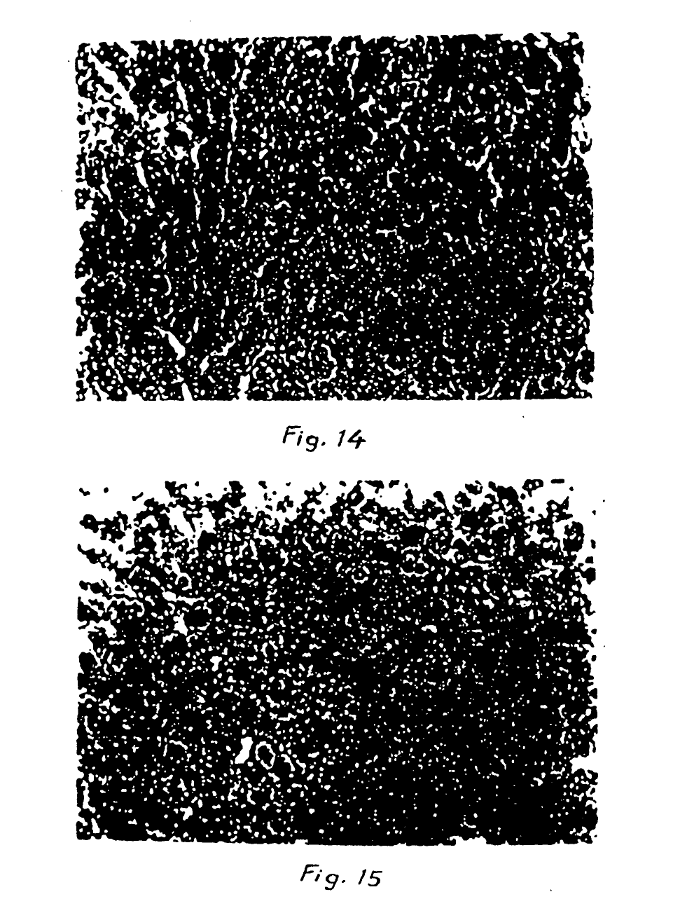

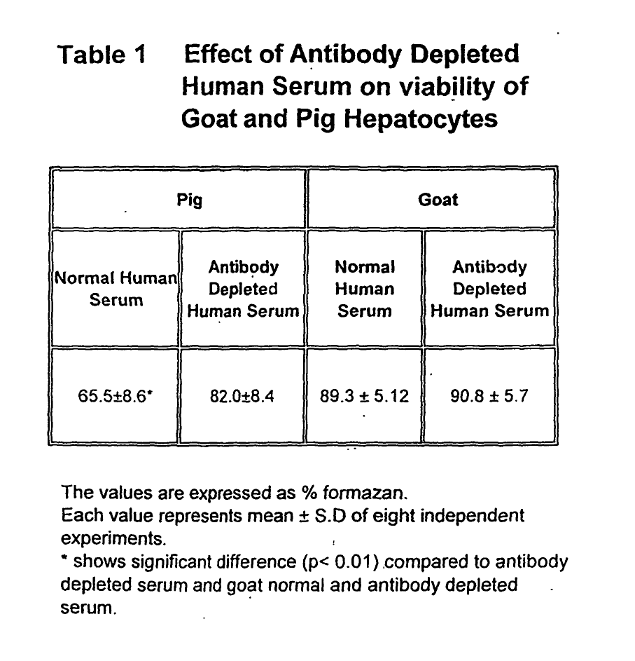

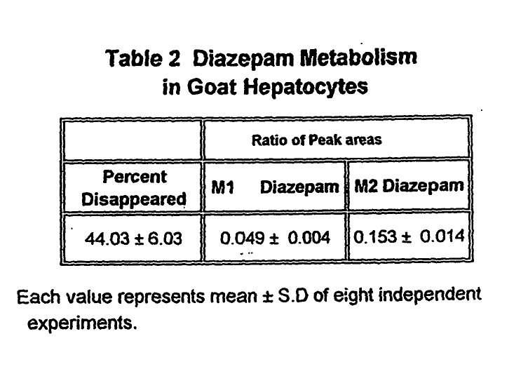

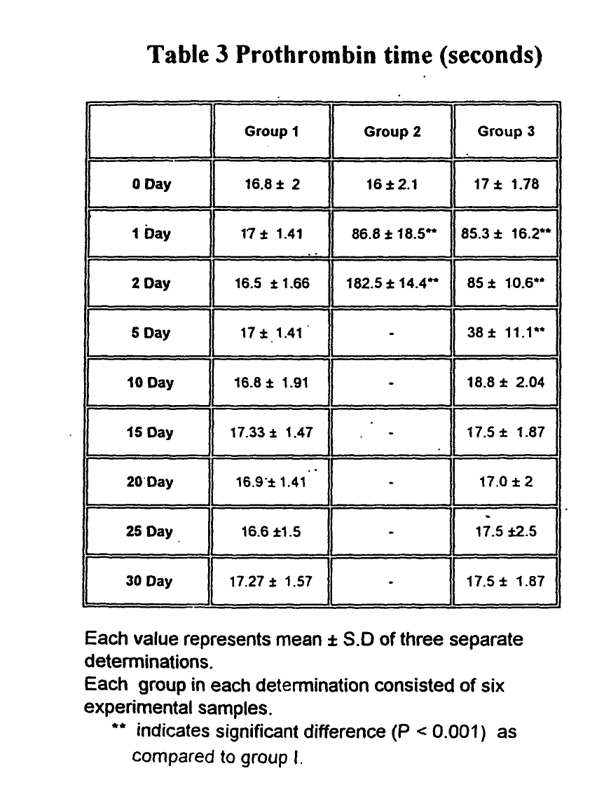

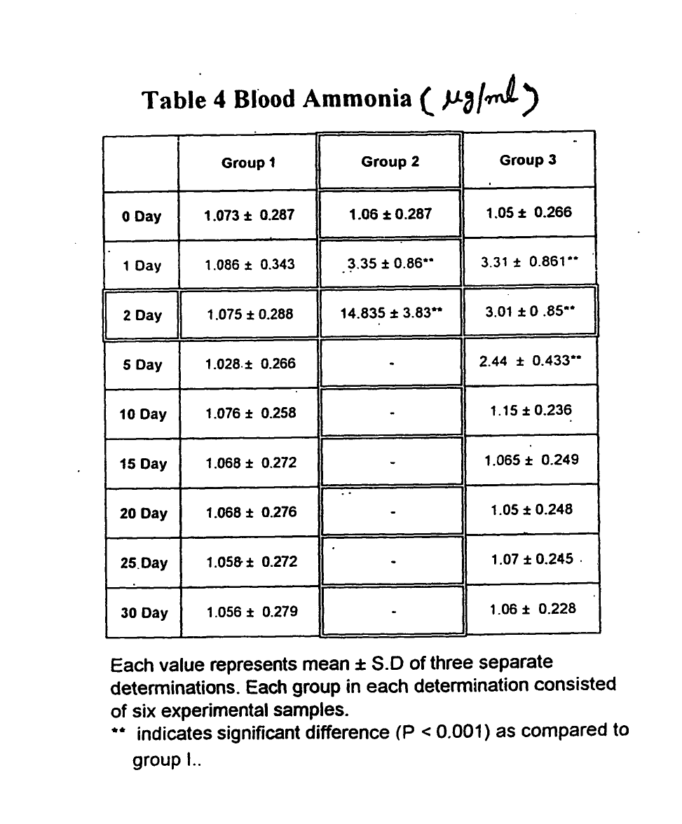

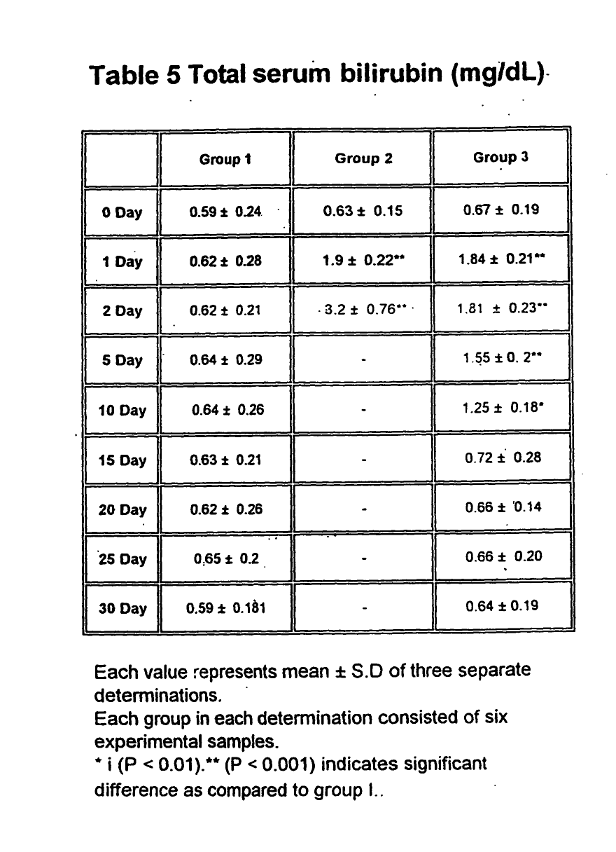

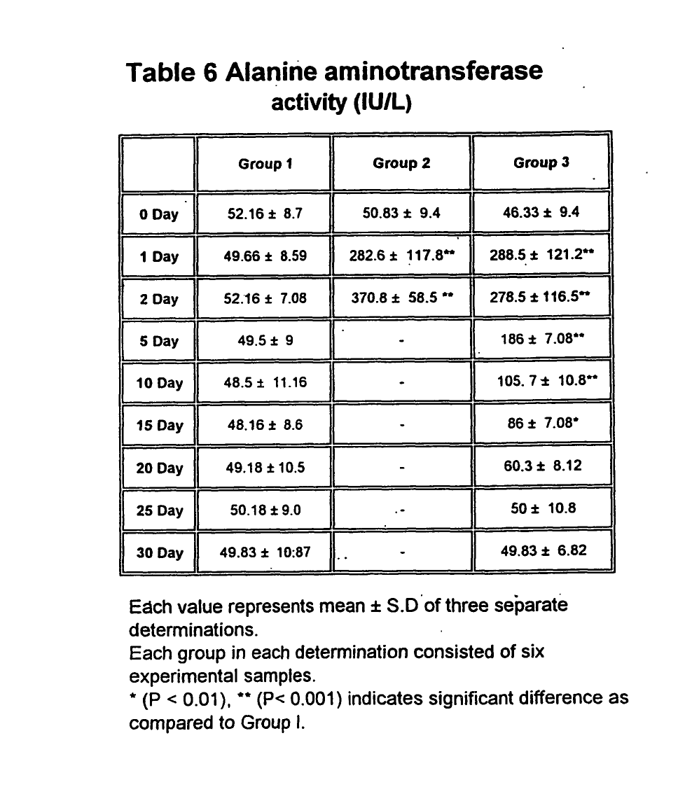

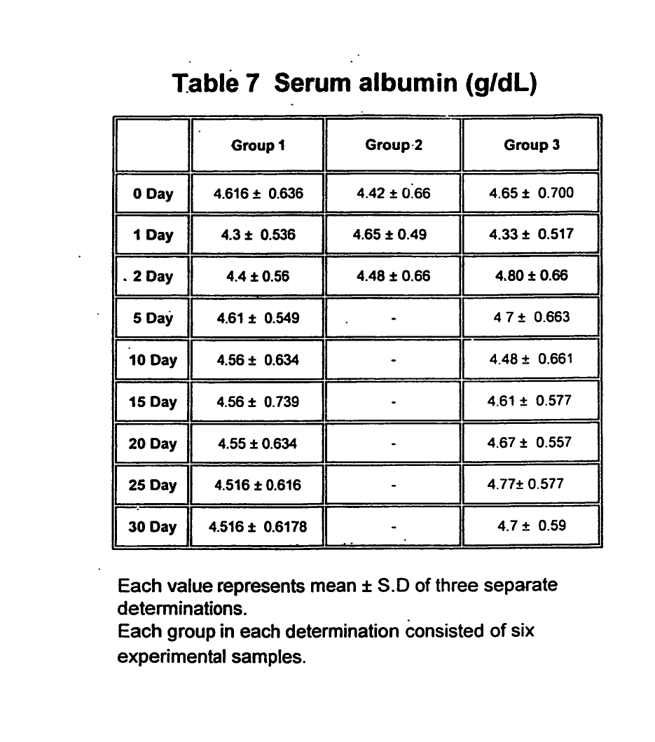

The Group I animals (Controls) showed normal liver morphology (FIG. 10). After 24-36 hours of D-galactosamine injection, histopathology of the liver showed massive necrosis (FIG. 11). Group III animals, on day 5 showed diffuse and extensive necrosis (FIG. 12). On day 10, showed occasional small foci of necrosis (FIG. 13). On day 15, focal cell clusters were seen within the sinusoids (FIG. 14). After one month of the hepatocyte transplantation, animals showed essentially normal morphology (FIG. 15).

1-5. (canceled)

6: A process for characterization of hepatocytes cells in goat comprising:
isolating goat hepatocytes and characterizing the isolated hepatocytes by biochemical and molecular techniques to demonstrate the hepatic functions.

7: The process as claimed in claim 6, wherein said step of isolation is carried out in a laminar flow unit under aseptic conditions.

8: The process as claimed in claim 7, wherein the isolation of a single cell suspension is performed using collagenase digestion.

9: The process as claimed in claim 7, wherein the isolated cells are hepatocytes which demonstrate the hepatic functions.

10: The process as claimed in claim 7, wherein said hepatic functions comprise ureagenesis, detoxification and albumin synthesis.
